(19) United States
(12) Patent Application Publication
(10) Pub. No.: US 2016/0106604 A1
(43) Pub. Date: Apr. 21, 2016

(54) DEVICE COMPRISING AN ABSORPTION ARTICLE AND AN AIR-EXTRACTION DEVICE, AND METHOD OF OPERATION USING THE AIR-EXTRACTION DEVICE FOR INFLUENCING THE HUMID CONDITIONS IN AN ABSORPTION ARTICLE

(71) Applicant: EBERHARD TIMM GMBH, Buxtehude (DE)

(72) Inventor: Eberhard Timm, Buxtehude (DE)

(73) Assignee: Eberhard Timm GmbH, Buxtehude (DE)

(21) Appl. No.: 14/889,836

(22) PCT Filed: May 6, 2014
(86) PCT No.: PCT/DE2014/000229

§ 371 Date: Nov. 6, 2015

(30) Foreign Application Priority Data
May 6, 2013 (DE) 10 2013 104 659.2

(51) Int. Cl. A61F 13/84

(52) U.S. Cl. CPC A61F 13/84

(57) ABSTRACT

The invention relates to a device comprising an absorption article, and an air-extraction device for humid conditions prevailing in the region of the absorption article, for example a diaper, when worn by a person, and also comprising a flexible container, which is arranged in the humid region of the person's body, is provided with apertures in its wall and has air exchanged by way of a pump. A method of operation for improving the conditions in the absorption article by having air extracted by suction from the absorption article using the air-extraction device is also described.

The invention relates to a device comprising an absorption article and an air-extraction device for the humid conditions prevailing in the region of the absorption article, such as a diaper or sanitary pad, when worn by a patient, and to a method of operation for improving the conditions in the absorption article by air being extracted by suction from the absorption article using the air-extraction device.

Absorption articles in the form of diapers, diaper pants or pads containing an absorption substance, such as a superabsorbent, are commonly known. Diapers are used, for example, for babies and toddlers and for patients in health care and nursing facilities due to illness. Urine or stool incontinence, or inability to get to a toilet independently and in time any more for other reasons, are collectively and in a simplifying manner referred to as incontinence. Patients who suffer from incontinence and cannot change their diaper themselves due to illness, e.g. because they can no longer leave the bed on their own, often develop decubital ulcers. These pressure ulcers and bedsores develop particularly in the sacral region and on the femurs due to the pressure load resulting from bed confinement or lasting wheelchair confinement. Even though wearing the diapers that are indispensable for incontinent patients does not directly cause the ulcers, it promotes ulceration and has a negative impact on wound treatment. The reasons are high temperatures in non-physiological regions and intense moisture that develop due to sweating and from excrements with a highly acidic pH that are typical of urine and stool incontinence.

It is known from U.S. Pat. No. 5,515,543 that a garment such as a jacket can be ventilated inside by blowing in air. Conversely, U.S. Pat. No. 5,192,276 A relates to a device for eliminating smoke generated during electrocautery surgery in the form of a flat, flexible panel-shaped hollow body with a multitude of openings on its top side and an adhesive for fastening directly on a patient's skin on its bottom side.

Prior art relating to aspirating urine is known to include a great variety of approaches. For example, U.S. Pat. No. 8,241,262 B2 discloses a flexible pad for collecting and transporting liquid to a collection point adapted to be positioned under an incontinent patient or worn under an undergarment. Document WO2006/014240 A2 describes a urine aspiration and collection device. Urine is aspirated close to the body and extracted into a urine collection vessel. Another device for collecting and draining urine is known from DE4236097 A1, however without an extraction system. Similar to WO 2006/014240 A2, U.S. Pat. No. 7,018,366 B2 relates to a urine collection system. The garment that is shaped like a diaper or undergarment includes an intake manifold and a multitude of perforated tubes sandwiched between layers of material. When urine gets into the garment, it is absorbed through the holes by negative pressure and pumped via a tube into a bag. The prior art mentioned above that relates to the absorption or drainage of urine lacks an absorption article for absorbing the liquid and its object is to absorb liquid and not air, as in the present invention.

It is the object of the invention to improve or supplement the use of absorption articles such as diapers in that the conditions in the region of the affected skin areas is favorably influenced in terms of humidity and temperature to reduce the risk of decubital ulceration, facilitate healing or reduce their healing-impairing effect.

This object is achieved, according to the invention, by the subject matter described in claim 1. Advantageous embodiments are the subject matter of the dependent claims or described below.

The air-extraction device used in conjunction with the absorption article ensures that contaminated air is extracted from the humid climate area near the patient's body and discharged to the outside. The replenishing air volume has an additional cooling effect. For this purpose, the air-extraction device comprises a ventilation system to absorb contaminated air from the humid climate area near the patient's body across an area distributed along the ventilation system and to discharge it to the outside.

The air-extraction device is permanently integrated into the absorption article or is slid or inserted into or placed onto it.

The air-extraction device is not meant to absorb liquids, except for condensate from the air that could form in the air intake process. However, it is preferred that there is no condensation in the air-extraction device, or if there is any, the same is absorbed by subsequent, not completely satiated are such that no liquid enters the air-extraction device throughout its runtime. For reliable prevention of liquid absorption, the air-extraction device and the absorption article are in particular fitted to a body and disposed relative to one another that the apertures of the air-extraction device are located in perpendicular direction (by height only) above the sites where stool and/or urine are collected in the absorption article, especially in sitting or lying position, to prevent liquid from being absorbed into the air-extraction device that is either absorbed directly at the excretion site by the absorption material or succumbs to gravity and moves away from the apertures.

The absorption article is in particular a diaper, also in the form of a diaper pant, or a pad. Such diapers can for example be designed as a fabric that can be folded in the crotch and has connecting lateral parts, or a diaper pant in the form of briefs. The absorption article preferably contains a superabsorbent that absorbs polar liquids, such as water or urine. Superabsorbents are in particular polymers that are capable of absorbing a multiple of their own weight of polar liquids. These are primarily water or aqueous solutions. When absorbing the liquid, the superabsorbent swells up and forms a hydrogel.

In addition to the suction mode, it is also possible to temporarily switch to another mode of operation and blow air into the air-extraction device to drive the contaminated air in the humid climate area near the patient's body away and to discharge in this manner. Both operating modes, suctioning and blowing, are applied in combination, e.g. with a time offset or at intervals. It is however preferred to schedule longer running times for suctioning off than for blowing in air.

The air-extraction device comprises a ventilation system to aspirate contaminated air across an area along the ventilation system from the humid climate area near the patient's body or blow the air into the humid climate area and then discharge it to the outside.

Continuous extraction of air from the inside of the absorption article leads to continuous replacement of the air volume inside the absorption article by uncontaminated, cooler air from outside, e.g. preheated air from the inside of the bed. Stored heat and moisture, are preferably removed, most of all, from the areas of the absorption article that are close to the body, and the affected area is cooled. The air-extraction device perceivably improves the climate in the affected areas. The conditions in the diaper region on the body are thus freed from harmful discomforts such as excessive humidity and heat, and the air-extraction device can therefore contribute to the prevention and treatment of decubital ulcers.

The air-extraction device has another prominent function in addition to discharging humidity. It can reduce the temperature near the surface of the skin. This is particularly true of the sacral region and the insides of the thighs where temperature rises above the normal body temperature when the patient is lying on the back or on the side. The cooler air that flows through the air-extraction device form air intake to the discharge of air via the tube connection dissipates high temperature or does not let it occur at all.

The air-extraction device includes a flexible container with a connection for a pump, an air-conducting interior space and a substantially air-impermeable surface with substantially air-impermeable zones and air-permeable zones with apertures to extract air from the environment through said apertures into the interior space of the container and through the connection. The apertures provide inlet faces for air. The interior space is preferably at least partially filled with filler material. The substantially air-impermeable surface may for example be a film, such as a plastic film.

The flat flexible container can be designed as a bag with an top film layer and a bottom film layer between which a filler material is disposed. The bag or container can also have the shape of a rod, a tube, or multiply branched tube.

The filler material is used to conduct the air and to keep the top and bottom film layers apart. The top and bottom film layers are preferably arranged parallel to one another. The two substantially air-impermeable film layers can also be made of one film layer that is folded over on one edge. One or both of the substantially air-impermeable film layers comprise apertures for air inlet and air outlet. This includes that said apertures are located near the edges of the film layers where the film layers are joined. The apertures can differ in number and shape and be provided on the outer surface only (away from the body) or on the inner surface only (facing the body) or on both sides alike.

According to another embodiment of the invention, the joints on the edges of the film layers and optionally the ribs where the film layers and the fabric or fleece layer on top of them are joined are formed by gluing or bonding.

According to another embodiment of the invention, the film layers are connected to one another in a strip-like manner, alternately from the one and the other longitudinal edges at a spacing from one another and perpendicular to the longitudinal edges of the container, wherein ribs are formed which, starting from the one respective longitudinal edge and at the opposite longitudinal edge such that a passage remains open which forces the extracted air to flow in a meander-like fashion. The strip-shaped joining of the film layers creates parallel chambers through which the extracted contaminated air must flow in a meander-like fashion and which enable wide-area extraction.

The filler material is preferably an elastic mat. The filler material is preferably air-permeable and/or leaves passages in the container open that serve as a ventilation system such that the air can be extracted from a wide area across the bag. It is also possible that the filler material comprises zones that are less air-permeable and zones that are more air-permeable to form a ventilation system. The purpose of the ventilation system is to distribute the air flow across the inlet surface.

At the same time, the filler material keeps the substantially air-impermeable surface, e.g. in the form of a top and bottom film layer, at a distance, preferably even when the weight of the body is fully or partially resting on it. Polyester fiber mats, like the ones that are used as coarse filter mats, can be used, for example, as the filler material. The elastic mats preferably have a progressive depth structure, i.e. they have a high initial deformation capacity which decreases with increasing compression. This means that they can be compressed easily at the start but then will not allow further compression above a specific measure under the body weight (e.g. related to 60 kg), such that they will remain air-permeable even in the compressed state. In addition to a synthetic fiber fleece, the filler material can also be a foamed plastic, for example.

According to another embodiment, the container of the air-extraction device can be made using air bubble films (“bubble wrap”). Air bubble films comprise at least one cover film and a second layer into which a multitude of air bubbles are incorporated at a regular spacing. Especially when air bubble films are folded such that the air bubbles face one another, an interior space is created that will conduct air even under pressure (as applied by the weight of a recumbent person). The multitude of air bubbles then forms the filler material.

According to another embodiment, the air-extraction device has an elongated shape and can be placed around the body in the hip region either under or on, preferably under, the absorption article, such that the zones of the air-extraction device without apertures are located in the back area and the zones of the air-extraction device with apertures are located in the ventral space at the level of the abdomen, preferably below the waistline,

wherein the connection is disposed in the starting section and the zones of the air-extraction device with apertures are disposed in the end section of the elongated air-extraction device. It is preferred that the belt-like air-extraction device has a length of 50%, particularly preferred of 65 to 95% of the girth and a width of 2 to 12 cm, in particular 4 to 8 cm. In the back region, the air-extraction device is used for cooling. Stored heat can accumulate in the absorption article, in particular in the areas compressed by body weight, resulting in sweating and additional strain on the skin.

According to another embodiment of the invention, the container is surrounded by an air-permeable fabric or fleece layer, which is further preferably joined on the edges with the film layers. The fabric or fleece layer prevents the skin from contacting the container surface.

According to another embodiment of the invention, the surface area of the container is shaped to match a diaper or pad and a larger length of it can be at least partially wrapped around the body. The air-extraction device is in particular designed such that the outlet in the form of a pump connection is not disposed on the container directly where the body comes to rest on, but, for example, on the abdominal side.

The connection is configured as a suction port for extracting the contaminated air from the container and optionally as an air inlet, such that the contaminated air that is suctioned into the container can be discharged to the outside using a pump in suction operation, and optionally air from the environment can be blown into the bag using said pump in compressor operation and discharged to the outside through the apertures.

According to an embodiment of the invention, the connection of the air-extraction device is connected to a pump via a tube connection. The amounts of air that are blown in or pumped out are small, e.g. 0.1 to 1 liters per minute, preferably 0.2 to 0.5 liters per minute.

The patient cannot perceive this mechanically or acoustically, and there is no risk of hypothermia. The pump, which is also called a compressor with reference to air as its working medium, can be equipped with an odor filter for suction operation. Activated charcoal is particularly suitable as an odor filter. According to another embodiment of the invention, the suction pump is located in a sound-proofed, handy box. A diaphragm pump is preferably comes into consideration as the pump. A temperature or humidity sensor can control the pump or pump output.

The air-extraction device can be inserted into, slid into, or placed onto the absorption article as a separately manageable unit or is an integral part of the absorption article. According to another embodiment of the invention, the air-extraction device is commercially available as an accessory for disposable absorption articles or integrated into absorption articles.

The air-extraction device operates in conjunction with a method according to the invention for influencing humid and warm conditions around an absorption article that fits tight to a patient's body, especially with a superabsorbent, such as a diaper, and is characterized by extracting humid, warm, contaminated air from the humid and warm climate area near the patient using an air-extraction device, wherein the contaminated air is absorbed in the humid climate area and discharged from there to the outside by the air-extraction device.

Decubitus affects about 3 million people in Germany. Most of them are incontinent. Decubitus is currently treated or prevented using padded mats as pads or by adhesive pads and special dressings. Skin transplantation is performed in severe cases.

The device according to the invention was used in a series of tests on a small group of patients under medical supervision and with the patients' consent.

The air-extraction device had the shape of a tube (approx. 5×85 cm) with an air inlet in the one end section and extraction on the other end of the tube, and it was inserted into the diaper and wrapped around the body with it. Air was extracted at a pump output of 0.25 L/min. The back region was free of apertures. When applied regularly, the above device prevented the occurrence of decubitus in almost all new patients admitted with incontinence but without decubitus. Wherever decubitus did occur in its weakest form, it was successfully treated in a short period of time using adhesive pads. Patients with decubitus showed unexpectedly good outcomes. Applied together with wound treatment, the wounds became smaller or closed, and no new wounds occurred. This was attributed to the fact that humid air enriched with acids was continuously extracted from the inside of the diaper and replaced with fresh air, but also to the fact that the temperature inside the diaper and particularly on the back was reduced by cooling to healthy temperatures and that sweating was minimized. The attending physician was surprised that the device was able to prevent decubitus in all problem zones, such as the sacrum and hip bones, and that the wounds even of people over 70 years of age could be healed.

The invention will be explained in greater detail with reference to the drawings. Wherein:

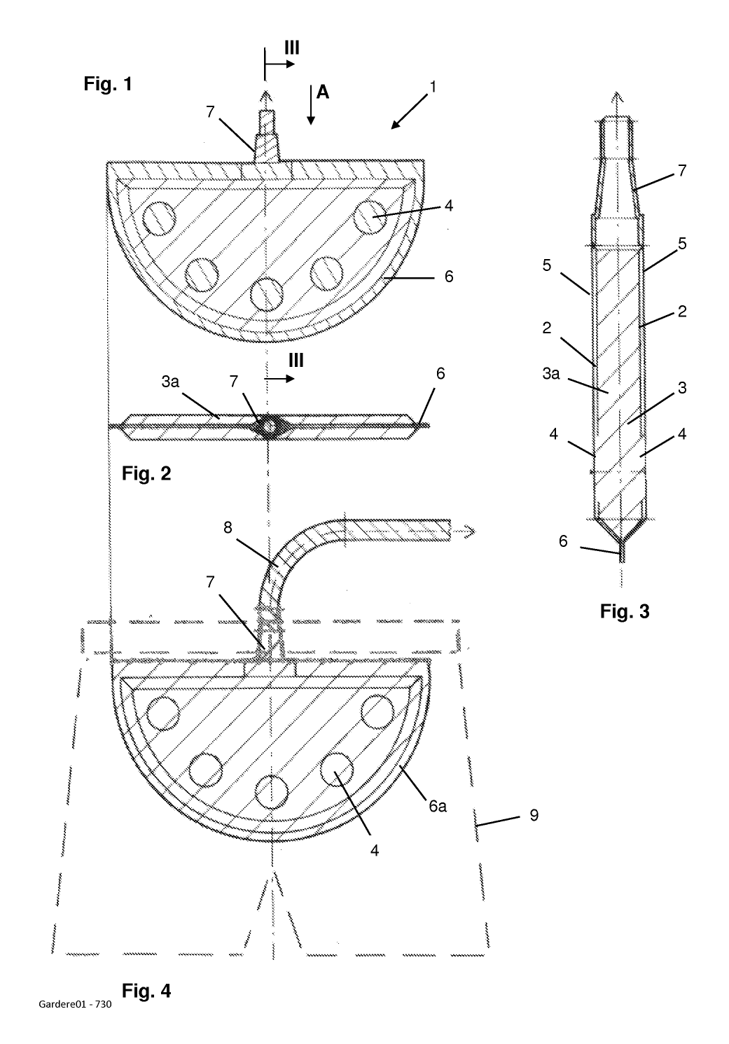

FIG. 1 shows a top view of a bag-like, flat air-extraction device disposed within the humid climate of an absorption article, such as a diaper, and with the help of which air is extracted from said humid climate area and discharged to the outside via a connection;

FIG. 2 shows a view A of the air-extraction device from the connection;

FIG. 3 shows an enlarged sectional view of the air-extraction device along the line III-III according to FIG. 1;

FIG. 4 shows the air-extraction device in a sketched out diaper pant with an extraction tube connected to the connection;

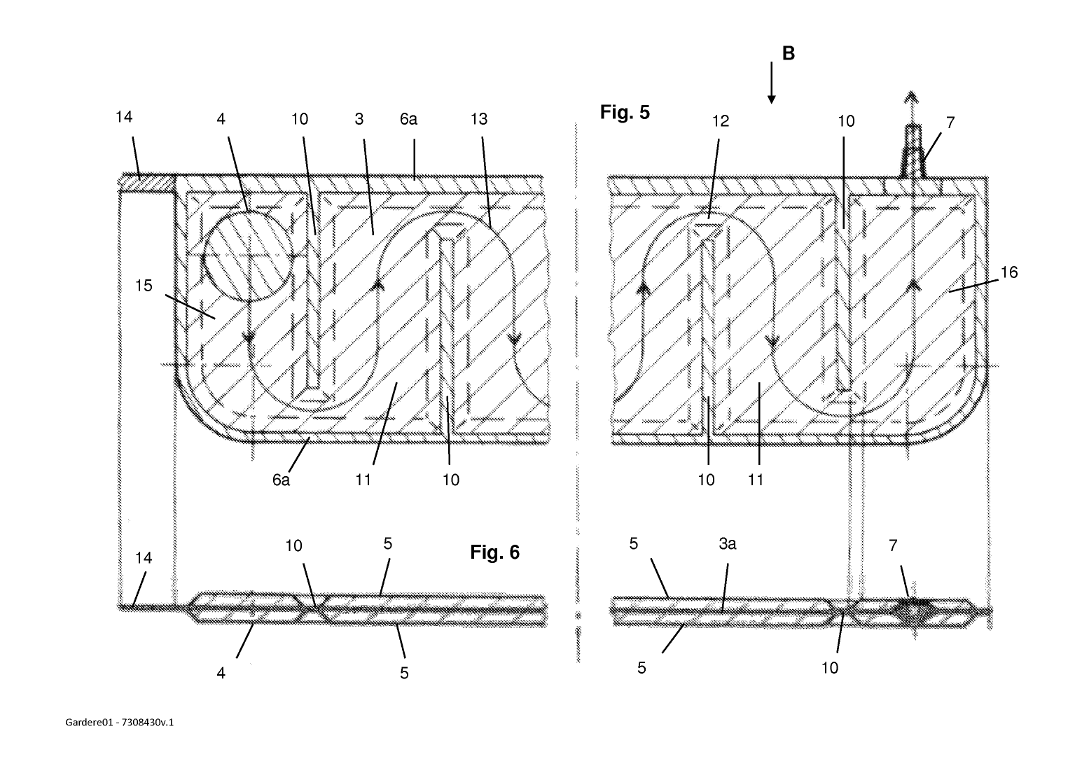

FIG. 5 shows a variant of the air-extraction device in the form of an elongated bag that can be placed around a patient's body and comprises chambers which force the extracted air to flow through the bag in a meander-like fashion;

FIG. 6 shows a sectional view of the air-extraction device according to FIG. 5;

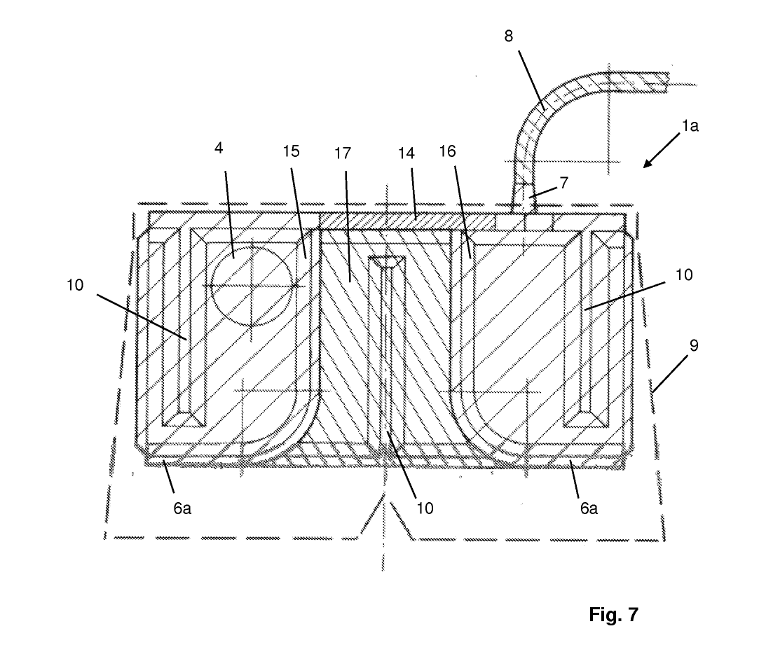

FIG. 7 shows a sketched out diaper pant in which the air-extraction device according to FIGS. 5 and 6 is located in a permanently integrated form, wherein the patient's hip or body is not shown; and

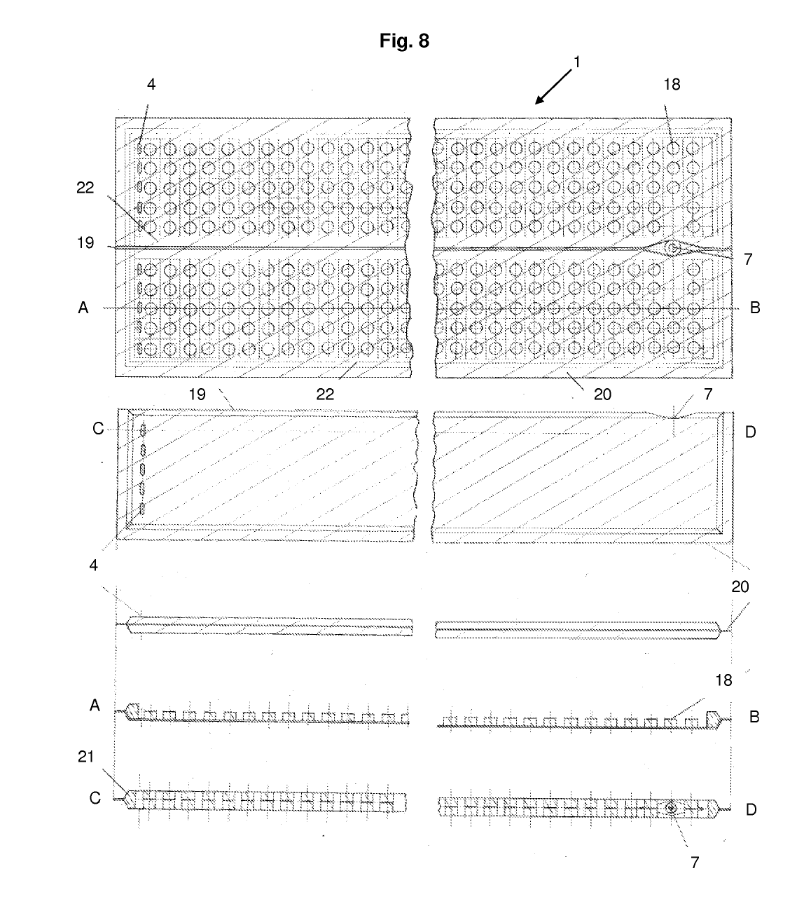

FIG. 8 shows an air-extraction device that can be produced by injection molding comprised of two foldable half-shells.

The air-extraction device according to FIGS. 1 to 4 is a container in the form of a bag (1) with an outlet fitting (7) for connecting a flexible tube end. The bag (1) according to FIG. 1 consists of two film webs (2). These film webs (2) form a barrier layer that prevents the passage of air in places where it is not perforated. The film webs (2) are kept at a spacing from one another by a filler material (3) inserted in between. This creates an interior space (3a) in the bag (1). The filler material (3) is an air-permeable, porous, and compressible mat that will however remain air-permeable when compressed. The film webs (2) comprise apertures (4) in the form of a perforation. The design of the perforation is completely free-form. The apertures (4) may also be square. The entire bag (1) is also coated with an air-permeable non-irritant fleece layer (5). The film webs (2) and the fleece layer (5) are bonded together on the edges (6).

An extraction fitting (7) is attached to the bag (1). An extraction tube (8) is connected to it and also connected to a suction pump not shown here. It can be housed in a box together with an odor filter. The air extraction volume can be about 0.4 liters per minute.

The bag (1) is either offered as an accessory or incorporated into the diaper during production. It is the purpose of the bag to extract contaminated air but no liquid from the diaper. The contaminated air is sucked into the interior space (3a) of the bag. The pump extracts this contaminated air that has entered the interior space (3a) of the bag via the tube connection (7) and tube (8). This results in dehumidification and, first of all, cooling. This counteracts the formation of decubitus and facilitates the treatment of an existing decubitus. The patient does not hear the pump and the air flow.

FIG. 4 shows the bag (1) in such a size that it is slid into a diaper pant (9). The extraction tube (8) is connected to the extraction fitting (7).

FIG. 5 shows a variant (1a) of the bag (1). This bag (1a) has a length such that it can be placed around the patient's body. The bag (1a) of course sits in a diaper that can be a diaper pant (9). The bag (1a) is shown unwrapped for the sake of explaining its structure. The bag (1a) has in this case the shape of a flat bag looking like a ribbon. Like in FIGS. 1 to 4, a filer material (3) is coated on both sides with film webs (2) and an air-permeable, non-irritant fleece (5) (FIG. 6). The ribbon form yields parallel longitudinal edges (6a) that are bonded or glued together. The film webs (2) comprise apertures (4) disposed in the first chamber (11) that is farthest away from the outlet.

The film webs (2) are alternately bonded or glued at a spacing from one another as strips coming from the one or the other longitudinal edge (6a) and perpendicular to the longitudinal edges (6a) of the bag (1a). This pattern creates ribs (10) that form adjacent chambers (11). The ribs (10) that extend from the respective longitudinal edges (6a) do not fully extend to the respective opposite edge (6a). They always leave a passage (12) open. This forces the contaminated air to be extracted via the extraction fitting (7) into a meander-like flow pattern (13). The contaminated air that has entered the bag (1a) is thus entirely extracted therefrom.

A closure patch (14) can be used to close the ring-shaped bag (1a).

FIG. 7 shows the bag (1a) slid into a diaper pant (9). The diaper pant (9) is once again shown in outline only and without the patient's body. One can see the left and right ends (15, 16) of the bag (1a) positioned at the front. In the center, the central part (17) of the bag (1a) is visible that is located under the patient's body, which is not shown.

The bag (1, 1a) is either offered as an accessory (1) or incorporated into the diaper during production (1a). The purpose of the Bag (1a) corresponds to that of the bag (1) according to FIGS. 1 to 4. The bags 1 and 1a will be disposed with the diaper after use and can thus be disposable items.

The method for discharging the contaminated air is based on the following characteristics. The used bag (1, 1a) does not necessarily have to be slid into the absorption articles. It can be incorporated into the absorption article during manufacturing, placed on the absorption article, or inserted between the skin and the absorption article.

FIG. 8 shows several sectional views of an air-extraction device as can be produced by injection molding from a flexible plastic material. Only the two end sections are shown, respectively. The top drawing shows the container in the form of two unfolded shells that can be collapsed using a hinge (19). Below is a top view and a lateral view of the functional air-extraction device with the two shells folded together. A closure strip (20) adjacent to the hinge (19) ensures coherence of the two shells. In the folded state, opposing spacer knobs (18) keep the two shells apart and open air flow paths between the spacer knobs (18). Edge bars of different height (21, 22) enclose and seal the interior space. The extraction fitting (7) is inserted into the edge bar (22) in the right end section in the top view.

Apertures (4) for taking in air are disposed on the other end. A zone that is air-impermeable on the outside and meant to be placed onto the skin in the back region stretches between the two ends. The flexible air-extraction device is 85 cm in length and 13 cm in width. Section A-B shows a section through a shell and section C-D shows a section through both shells in the closed state.

### LIST OF REFERENCE SYMBOLS

(1) Bag/container
(1a) Bag/container integrated into the diaper
(2) Top and bottom film/film webs as walls of the bag
(3) Filler material between the film webs
(3a) Interior space of the container or bag
(4) Apertures/perforation
(5) Air-permeable fleece
(6) Bonded edges
(6a) Bonded longitudinal edges
(7) Connection/extraction fitting
(8) Extraction tube
(9) Absorption article/diaper pant
(10) Ribs
(11) Chambers
(12) Passage
(13) Meander-like air flow path
(14) Closure strip
(15) Left end of the bag (1a)
(16) Right end of the bag (1a)
(17) Rear central part of the bag (1a)
(18) Spacer knobs
(19) Hinge
(20) Closing edge
(21) Edge bar
(22) Semi-high edge bar

1. A device including an absorption article, a pump, and an air-extraction device in, under, or on the absorption article,
wherein the air-extraction device comprises:
a flexible container,
an air-conducting interior space,
a connection, and
a surface with substantially air-impermeable zones, the air-permeable zones being in the form of apertures to extract air from the absorption article through the apertures via the connection into the air-conducting interior space of the flexible container,
wherein the air-extraction device is a separately manageable unit and slid into or inserted into, or placed onto the absorption article, and
wherein the air-extraction device is connected to the pump via the connection, and the pump configured to extract contaminated air from the flexible container via the connection by suctioning.

2. The device according to claim 1, wherein the substantially air-impermeable surface comprises at least a top film and a bottom film that form a barrier layer for air passage and at least one of the films comprises apertures for air passage.

3. The device according to claim 1, wherein the flexible container is partially or completely provided with an air-permeable fabric or fleece layer which preferably covers the top film and the bottom film, and is further preferably joined with the top and bottom films on one or more edges.

4. The device according to claim 1, wherein an elastic, air-conducting filler material keeps at least one set of opposite surfaces, and in particular the top film and the bottom film, at a spacing from one another.

5. The device according to claim 1, wherein the air-extraction device further comprises a tube for connecting the pump to the flexible container, wherein the pump preferably extracts air through an odor filter.

6. The device according to claim 1, wherein a surface area of the flexible container is shaped to match the absorption article or is optionally wrapped around a body.

7. The device according to claim 1, wherein the flexible container of the air-extraction device comprises adjacent first and second longitudinal edges, which are alternately interconnected at a spacing from either the first or the second longitudinal edge, wherein ribs are formed which, starting in turns from the first respective longitudinal edge and ending sufficiently distant from the second longitudinal edge, and a passage remains open which forces air in the interior space to adopt a meander-like flow pattern.

8. The device according to claim 7, wherein joined edges and the ribs, where film webs, and optionally fabric and fleece layers above the same, are interconnected, and are formed by gluing or welding.

9. The device according to claim 1, wherein the air-extraction device is configured for one or more of the group that includes sliding into a diaper pant, inserting between skin and the absorption article, and placing onto the absorption article.

10. The device according to claim 1, wherein the absorption article includes a superabsorbent that absorbs polar liquids such as water or urine.

11. The device according to claim 1, wherein the air-extraction device and the absorption article are configured to fit to a body, and arranged relative to one another so that the apertures of the air-extraction device are located in a substantially perpendicular direction above areas where stool or urine are collected in the absorption article, to prevent absorption of liquid.

12. The device according to claim 1, wherein the air-extraction device does not soak in liquids other than condensate from the air.

13. The device according to claim 1, wherein the air-extraction device has an elongated shape, and can be placed around a body in a hip region, either in, under, or on the absorption article.

14. The device according to claim 1, wherein the absorption article is at least one or more from the group that includes a disposable absorption article, a diaper, and a sanitary pad.

15. A method for influencing humid conditions in an area of an absorption article positioned adjacent a person's body, such as a diaper, including:
extracting contaminated air from the absorption article using an air-extraction device designed as a separately manageable unit in, under, or on the absorption article, the air-extraction device comprising:
an air-conducting interior space with air-impermeable and air-permeable zones formed by a flexible container, and
a connection,
wherein the extracting of the contaminated air is from areas of the absorption article through the air-permeable zones into the interior space of the flexible container and further through the connection using a pump in a suction mode, such that the contaminated air is discharged from the flexible container via the connection by suctioning.

16. The method according to claim 15, wherein the pump extracts the air at a rate of 0.1 to 1 L/min (at 20° C. and 1013 mbar).

17. (canceled)

18. The device according to claim 1, wherein zones of the air-extraction device without apertures are positioned adjacent a back area of a person.

19. The device according to claim 1, wherein zones of the air-extraction device with the apertures are positioned adjacent a ventral space of a person at a level of an abdominal or pelvic region of the person.

20. The device according to claim 1, wherein the connection is disposed in a starting section and zones of the air-extraction device with the apertures are disposed in an end section of the air-extraction device having an elongated shape.

21. The device according to claim 1, wherein the air-extraction device has a length of at least about 50%, and, preferably, from about 65% to about 95% of a person's girth using the device.
